(19) United States
(12) Patent Application Publication
(10) Pub. No.: US 2013/0150684 A1
(43) Pub. Date: Jun. 13, 2013

(54) System and Method for Detecting, Recording, and Treating Persons with Traumatic Brain Injury

(71) Applicant: Jason Ryan Cooner, Birmingham, AL (US)

(72) Inventor: Jason Ryan Cooner, Birmingham, AL (US)

(21) Appl. No.: 13/760,797

(22) Filed: Feb. 6, 2013

Related U.S. Application Data
(63) Continuation-in-part of application No. 13/219,605, filed on Aug. 27, 2011.
(60) Provisional application No. 61/595,195, filed on Feb. 6, 2012.
(60) Provisional application No. 61/646,390, filed on May 14, 2012.
(60) Provisional application No. 61/719,499, filed on Oct. 29, 2012.
(60) Provisional application No. 61/724,676, filed on Nov. 9, 2012.

(51) Int. Cl. A61B 5/11; A61B 3/11; A61B 3/14; A61B 5/0205; A61B 5/145; A61C 5/14; A61B 7/00; A61B 5/00; A61B 5/026

(52) U.S. Cl. CPC A61B 5/1101; A61B 5/6803; A61B 5/7246; A61B 5/7282; A61B 5/4064; A61B 5/002; A61B 5/0022; A61B 3/112; A61B 3/14; A61B 5/0261; A61B 5/0042; A61B 5/14517; A61C 5/14; A61B 5/4875; A61B 5/4094; A61B 5/746; A61B 5/4088; A61B 5/4082; A61B 7/00; A61B 5/0205
USPC 600/301; 600/595

(57) ABSTRACT

A system and method for detecting information related to traumatic brain injury is presented, comprising a skull cap, adapted to be worn by an individual, and a sensor array coupled to the skull cap. The sensor array comprises one or more multi-axial accelerometers, adapted to measure linear, rotational, and/or angular forces, one or more gyroscopes, and a sensor array computer, coupled to the accelerometers and gyroscopes, including a processor for receiving data from the accelerometers and gyroscopes pertaining to the individual's vital, and a memory for storing data. The sensor array computer is adapted to compare the data to historical data for the individual to determine if a traumatic brain injury has occurred, wherein the historical data includes information relating to the frequency of injuries to the individual's head over a given time period, and to calculate acceleration from linear forces. The headgear preferably comprises a plurality of layers.

### RELATED APPLICATIONS

The present application claims priority to U.S. Provisional Application Nos. 61/595,195, filed Feb. 6, 2012, 61/646,390, filed May 14, 2012, 61/719,499, filed Oct. 29, 2012, and 61/724,676, filed Nov. 9, 2012, all of which are incorporated herein by reference. Additionally, the present application claims priority as a continuation-in-part application of U.S. Non-Provisional application Ser. No. 13/219,605, filed Aug. 27, 2011, which itself claims priority to U.S. Provisional Application Nos. 61/378,494, filed Aug. 31, 2010, 61/388,186, filed Sep. 30, 2010, and 61/453,197, filed Mar. 16, 2011, all of which are incorporated herein by reference as well.

### BACKGROUND OF THE INVENTION

There are only a few biomarker test mechanisms in use today that can be used to detect levels of cortisol in body fluids. One certain technique uses material as the sensor that is encoded with a particular molecular structure to target identifying the molecules of interest when certain fluids are exposed to or passed through the material. Body fluid samples specifically mentioned for this sensor test are saliva and urine. When these fluids are exposed to the material surface or passed through the material, molecules of interest (in this case cortisol molecules) are captured by the sensor. This invention was disclosed in the U.S. Pat. No. 6,833,274, which issued on Dec. 21, 2004 to David Lawrence, and was titled “Cortisol Sensor.” However, this test is not easily repeatable as it requires the material sensor to be flushed or cleaned of the molecules it captures between taking measurements on a sample.

Another technique describes an antibody functionalized interdigitated u-electrode (IDuE) based impedimetric cortisol biosensor, which is described as working with saliva as well but takes about 40 minutes to render accurate measurements.

Another technique uses nanotechnology to create a small electronic biosensor that can detect cortisol in fluids, and mentions blood and urine as the body fluids to use for this measurement. This technique is being developed by Banyan Biomarkers in Gainesville, Florida, and can be performed in 5-10 seconds and can be implemented so that it is repeatable.

Although these techniques as well as all other cortisol sensor implementations currently available may be best suited for use in lab experiments and clinical practice, it would be advantageous to detect TBI, stress/fatigue, and dehydration while a person is playing sports or performing other physical activities where cortisol measurements may be desired to monitor for those conditions. In the scenarios, it would be advantageous to provide the biomarker sensor technology for and have a “wearable” low power cortisol sensor implementation that is repeatable without the person wearing the sensor to have to perform any operation to retest the cortisol measurements when desired. It would also be advantageous to perform the test in a manner that doesn't require blood, urine, or saliva as the source specimen for the sensor.

### SUMMARY OF THE INVENTION

A system and method for detecting information related to traumatic brain injury is presented, comprising a skull cap, adapted to be worn by an individual, and a sensor array coupled to the skull cap. The sensor array comprises one or more multi-axial accelerometers, adapted to measure linear, rotational, and/or angular forces, one or more gyroscopes, and a sensor array computer, coupled to the accelerometers and gyroscopes, including a processor for receiving data from the accelerometers and gyroscopes pertaining to the individual's vital, and a memory for storing data. The sensor array computer is adapted to compare the data to historical data for the individual to determine if a traumatic brain injury has occurred, wherein the historical data includes information relating to the frequency of injuries to the individual's head over a given time period, and to calculate acceleration from linear forces. The headgear preferably comprises a plurality of layers. The plurality of layers preferably includes an outer rigid layer, an inner rigid layer, and at least one softer middle layer in-between the outer layer and inner layer. The middle layer is preferably adapted to compress upon impact to the head of the individual and to expand to its original shape after the impact for maximum protection of the individual's head.

### BRIEF DESCRIPTION OF THE DRAWINGS

FIG. 1 is an example of one embodiment of the present invention, showing the sensor array attached to a skull cap to be worn on the head of an individual; and

FIG. 2 shows the connection of the sensor array in the present invention to a network computer for collection and analysis for data, and for notifying an injured individual or others about a detection of traumatic brain injury.

### DETAILED DESCRIPTION OF THE DRAWINGS AND PREFERRED EMBODIMENT

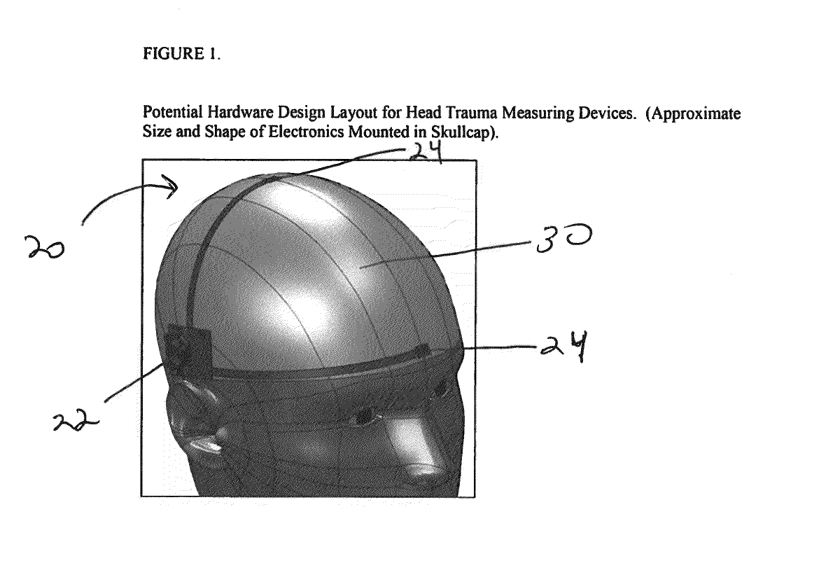

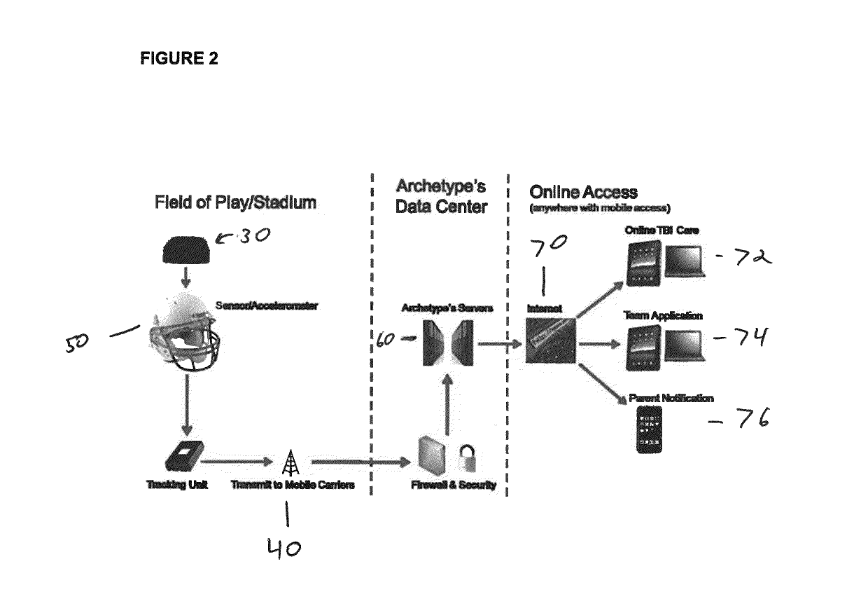

Description will now be given of the invention with reference to the attached FIGS. 1-2. It should be understood that these figures are exemplary in nature and in no way serve to limit the scope of the invention as the invention will be defined by the claims, as interpreted by the Courts in an issued US Patent.

It is known throughout the medical community that the hormone cortisol is contained within body fluids, and that measuring cortisol levels can allow one to monitor for traumatic brain injury (TBI), overall stress/fatigue, and dehydration in humans. For the purposes of this disclosure, we will focus on describing where the current biomarker technology is for detecting cortisol levels in the human body. In addition, we will discuss how we have further enhanced the technology of measuring linear and rotational forces to detect traumatic brain injury, as discussed in U.S. Non-Provisional application Ser. No. 13/219,605, filed Aug. 27, 2011, which is specifically incorporated by reference herein.

The medical neuroscience industry has been actively seeking to develop a technique that can actually detect traumatic brain injury (TBI) as it occurs. However, no previous attempts have resulted in any markers, bio or otherwise, that would conclusively detect the tissue damage as it occurs. We reviewed the injury in detail and assessed that TBI is caused by two primary events. Either the head is impacted and the brain is slammed against the inside of the skull causing the brain to bruise, or the head is impacted in a manner that causes brain tissue to shear, which results in tissue being torn.

After years of researching the issue, it finally became apparent that there was a single aspect of the injury that could be monitored in real-time in a non-obtrusive wearable manner that may accurately detect TBI as it occurs. That aspect is audio. We believe there are specific sound resonations that are produced during the brain bruising and/or shearing that could be listened for by a headgear design implemented with microphones and/or some other vibration detecting sensors. Considering that pretty much all human brain tissue is of a certain density, cerebrospinal fluids (CSFs) around the brain are of an identical chemical structure, and skulls are all made of course of bone, there should be some highly unique sound resonations that occur when tissue shears or the brain impacts the inside of the skull. In other words, if we put directional short range audio receivers and/or some other vibration detecting mechanism in a grid pattern around the skull that may be pointed toward the brain of the athlete that would listen for specific sound wavelengths and specific ranges of sound wavelengths that correspond to TBI events, one can hypothesize that this technique should result in a highly accurate indication that tissue damage consistent with TBI has occurred.

To better understand the types of audio and/or vibration signatures we want to isolate for TBI detection, we should better understand the physical properties of the injury and its mechanics. The brain itself is a gelatinous structure that is spongy in nature. The CSF that surrounds the brain is a more dense material that acts to hold the brain in place and serves as a cushioning mechanism for the brain inside the skull. The skull bone itself is somewhat jagged on the interior surface facing the brain. The medical community classifies TBI to be one of two basic types, focal or diffuse. Focal injuries are more consistent with cerebral contusion, or bruising of the brain, as the brain impacts the skull wall. Diffuse injuries (usually referred to as diffuse axonal injury or DAI) are where damage occurs across a wider area of the brain and are more consistent with the shearing of brain tissue and/or combinations of bruising and shearing. Both focal and diffuse TBI and the mechanics of how they occur are explained further in a concept called “coup contracoup”, or acceleration/deceleration injury. A coup injury occurs under the site of impact with an object, and a contracoup injury occurs on the side opposite the area that was impacted. Coup and contracoup injuries can occur individually or together. When a moving object impacts the stationary head, coup injuries are typical, while contracoup injuries are produced when the moving head strikes a stationary object. In a contracoup injury, the head stops abruptly and the brain collides with the inside of the skull. The coup injury may also be caused when, during an impact, the skull is temporarily bent inward, and impacts the brain.

When the skull bends inward, it may set the brain into motion, causing it to collide with the skull opposite side and resulting in a contracoup injury. The injuries can also be caused by acceleration or deceleration alone, in the absence of an impact. In injuries associated with acceleration or deceleration but with no impact, the brain is thought to bounce off the inside of the skull and hit the opposite side, potentially resulting in both coup and contracoup injuries. In addition to the skull, the brain may also impact the tentorium to cause a coup injury. Cerebrospinal fluid (CSF) is also implicated in the mechanism of coup and contracoup injuries.

One explanation for the contracoup phenomenon is that CSF, which is denser than the brain, rushes to the area of impact during the injury, forcing the brain back into the other side of the skull. If this is the case, the contracoup impact happens first. Contracoup contusions are particularly common in the lower part of the frontal lobes and the front part of the temporal lobes. A 1978 study found that the contracoup mechanism was responsible for most of the brain lesions such as contusions and hematomas occurring in the temporal lobes of injured individuals. Injuries that occur in body parts other than the brain, such as the lens of the eye, the lung, and the skull and other bones, may also be labeled “contracoup”. Due to this understanding, we believe that the focus of the audio patterns should be placed on listening for various contracoup audio signatures as they may be the determinate for the majority of actual TBI tissue damage.

Unlike focal brain trauma that occurs due to direct impact and deformation of the brain, DAI is the result of traumatic shearing forces that occur when the head is rapidly accelerated or decelerated. It usually results from rotational forces or severe deceleration. The major cause of damage in DAI is the disruption of axons, the neural processes that allow one neuron to communicate with another. Tracts of axons, which appear white due to myelination, are referred to as white matter. Acceleration causes shearing injury, which refers to damage inflicted as tissue slides over other tissue. When the brain is accelerated, parts of the brain having differing densities and distances from the axis of rotation slide over one another. This effect stretches axons that traverse junctions between areas of different density, especially at junctions between white and grey matter. Two thirds of DAI lesions occur in areas where grey and white matter meets. Lesions typically exist in the white matter of brains injured by DAI; these lesions vary in size from about 1-15 mm and are distributed in a characteristic way. DAI most commonly affects white matter in areas including the brain stem, the corpus callosum, and the cerebral hemispheres. The lobes of the brain most likely to be injured during DAI are the frontal and temporal lobes. Other common locations for DAI include the white matter in the cerebral cortex, the corpus callosum, the superior cerebral peduncles, basal ganglia, thalamus, and deep hemispheric nuclei. These areas may be more easily damaged because of the difference in density between them and the rest of the brain.

As the mechanics of the injury itself are considered, one can deduce that several observations can assist development of a system that can detect TBI as it occurs through audio signal and/or vibration analysis. First, the brain bouncing inside the skull as occurs in the “coup-contracoup” scenario could sound a lot like hitting your palm against a watermelon. Second, the shearing of brain tissue as it is pushed across the jagged internal edge of the skull wall may sound a lot like the beaching of an ocean wave. Third, microphones or other vibration based sensors could be placed in a grid arrangement around those regions to gain more sensitivity since most TBI damage seems to occur in the lower part of the frontal lobes and front part of temporal lobes. This could serve to also provide the specific location(s) and extent of the tissue damage, as well as assist in choosing better care, recovery, and reintegration programs for the patient. And last, human bone acts as a natural resonator/amplifier for audio and/or vibration waves. Due to this, the ideal location for listening to DAI at the base of the brain as well as potentially the entire brain cavity may be to place additional and potentially more sensitive microphones against the bone behind the ear pointing toward the interior of the brain. This may allow us to clearly record audio resonations and/or other vibrations specific to TBI throughout the brain cavity regardless of where they occur.

The cortisol sensor implementation disclosed in this filing is based primarily on collecting sweat from the human body for use as all other fluid sources mentioned in the above filings are not readily available during sporting or other physical activities. The ideal location on the human body to collect sweat is on the head of the individual as that is where more sweat is produced than any other part of the body in most people. The skull-cap and headband designs described in the applications on which the present Application claims priority are ideal designs for collecting the sweat for cortisol tests during physical activities. This is shown in FIG. 1, where the sensor array 20 is placed on a skull cap 30 to be worn on the head of an individual. The control panel 22 of the sensor array 20 could be placed anywhere around the circumference of the skull so that the control panel is at the back base of the skull. That way the design can minimize potential injury to the wearer of the headgear. Accelerometer placement points 24 may continue to be where the control panel is positioned, around the circumference of the skull to a point just above the ears as well as up the circumference of the skull to a point on the top part of the skull as primary location points for the accelerometers. Heat sensors should be placed near the temporal lobe in the headgear for best performance, and decibel sensors for detecting potential noise damage would be best located near each car for measuring noise.

The skull cap design could have ribs under the material made out of the skull cap material itself or by appending rubber ribs under the cap to direct sweat flow to the back base of the skull or other desirable collection point. This could serve several purposes. One, it would move sweat away from the front of the skull so that sweat is less likely to come down the forehead and into the eyes. Two, it could act to better ventilate the head for comfort and breathability. Three, it would optimize the collection of sweat from the head and maximize the likelihood of sweat samples being available for a cortisol sensor test when desired.

There may have to be a small reservoir that can open and shut as desired on both ends. This is desired to control collection and exposure of the sweat sample to the sensor, testing in a controlled manner, and clearing sweat from exposure to the sensor once the test is concluded. If the test is not repeatable, then the reservoir used for testing may only have to handle collecting a sweat sample and exposing that sample to the sensor when desired. The reservoir can be further benefitted by a collection mechanism located at or near the end of the described rib design above. This collection mechanism can work to maximize sweat fluid flow control to the cortisol sensor and can work in a few different manners. One could be that the collection mechanism is a sponge-like material that can constantly soak up the sweat as it is channeled to the collection area.

When a test is desired, then the sponge-like material can be mechanically squeezed to allow the sweat to enter the test reservoir. The sweat from the sponge-like material could also be released into the reservoir when desired via electrical current or some other excitation of either the sweat or the collection material itself. A small pump could also be incorporated. Since we can incorporate nano-technology, all the above implementations above can easily fit in a small form factor within the skull cap, headgear, or integrated into a helmet if desired. The flow control mechanisms can all be initiated by the electronic control panel, which can be used to coordinate using accelerometers, heat, and other sensors as well as radio frequency based requests to initiate the cortisol test. Another collection mechanism could be where the sweat that is directed to the back of the skull or located near the sensor can be allowed to flow out the back of the headgear under normal operation, and then when a cortisol test is desired, the mechanism can have the sweat redirected to a sponge-like collection material or directly into a chamber that may allow for proper distribution into the reservoir that contains the cortisol sensor as desired.

Rigid body acceleration of the skull is calculated by assuming a fixed distance between the three sensor pairs, and by assuming that the three are orthogonal to one another. The flexible skullcap makes this a risky assumption; relative position and orientation can both be seriously skewed by head size, shape of skull, hair style, and even the care with which the skullcap is donned.

The accelerometers, heat sensors, other sensors in the sensory array, or remote invocation from a computing interface over RF, could initiate a request to start testing cortisol levels, normally during physical activity. The cortisol sensor implementation could be started by the control panel in the headgear. The cortisol tests could be performed as a one-time scenario immediately or on a specified time delay from the incident of interest (i.e., head impact, heat threshold exceeded, coach wanting to know the stress/fatigue or dehydration levels of players).

The cortisol tests could be performed in a manner where the tests are polling the person's condition on a set time interval for TBI after a head impact or if TBI is suspected for a specified period. It may be desirable to start testing cortisol levels right after a 50 G-force impact to the head, and continue retesting the cortisol levels of the person every minute for a 15 minute window or until the physical activity has concluded. This would have the benefit of being able to sense when cortisol levels are increasing, and potentially at a level consistent with TBI, at the earliest possible time. As the levels are increasing, the sensor can send the measurement electronically to the control panel whereby logic either on the control panel, smartphone, or centralized computer facility can then dictate whether or not authority figures need to be notified of the injury. If levels are already exceeding levels consistent with TBI damage or dehydration, the system can send out text message (SMS) or email alerts to notify coaches, parents, athletic trainers, physicians, or other interested parties that a TBI may have occurred.

The cortisol test itself may be initiated by the accelerometers or heat sensors mounted to the skull cap, headband, or other part of the body in the manner described above so that the cortisol test is only performed when a significant measurable impact to the head or certain potentially dangerous heat levels have occurred. This is ideal for a “wearable” cortisol implementation from a power management perspective because it can use the accelerometers, heat sensors, remote RF invocation, or other sensors as a low-power repeatable “trigger” or initiator for the cortisol test. This would make the headgear last longer per battery, or for rechargeable batteries, longer per charge.

Figures and descriptions of the electronics as they would appear in the sensor array of the present application, the combination of the sensor array on the skull cap, embodiments and information of various batteries which could be utilized in the sensor array, and an embodiment of the present invention incorporating a multi-layered skull cap can be found labeled as FIGS. 2-9 and Tables 1 & 2 in U.S. Provisional Application No. 61/595,195, filed Feb. 6, 2012, which is specifically incorporated by reference herein.

There is another very good reason for having “triggers” to initiate the cortisol test. Since sweat is going to be used by the sensor in this implementation and the sensor may be made of potentially corrosive metals, to safeguard the sensor so that it can be used when desired, it is desirable to have another sensor type or another computer interface initiate the cortisol test during physical activities. This can help ensure that the sensor is working properly when desired. It can also ensure that the sensor isn't being exposed to undue contamination during sporting and other physical activities if used as a standalone sensor for TBI and other conditions, which should dramatically improve the real-world reliability of such a system.

The cortisol test can also be initiated via short range radio directly to the headgear if desired by a web interface connected to a smartphone for local signal relay, or it could be initiated by an application on the smartphone or other short-range radio frequency device running Zigbee, Bluetooth, Wi-Fi, or other standard communications protocol. The RF in use could be any “short range” radio including but not limited to Bluetooth, Zigbee, WiFi. The RF in use could also include GSM, CDMA, TDMA, 3G, 4G, LTE, WiMAX, or any other “long-range” wireless communications protocol in use. Such “long-range” mechanisms would likely have to work and be designed much like a cellular phone is today, and may include an LCD for display, SIM card for network permissions, a power source, antenna, and sensors that can further assist with the mechanism.

The same ribbing and collection implementation described for the skull cap and headband could be made into a mouthpiece, but due to the lack of available saliva while the person is engaged in high physical activity, this is not an ideal location. If however, a mouthpiece is used to contain the sensor, there may have to be a small reservoir that can open and shut as desired on both ends. This is desired to control collection and exposure of the saliva sample to the sensor, testing in a controlled manner, and clearing saliva from exposure to the sensor once the test is concluded. If the test is not repeatable, then the reservoir used for testing may only have to handle collecting a saliva sample and exposing that sample to the sensor when desired. In the mouthpiece design, the sensor may have to be powered by small lithium, NiCad, or other power source that can be embedded into the mouthpiece. The mouthpiece test can be initiated by local RF from the skull cap equipped with accelerometers for use as a low-power repeatable “trigger” or initiator for the cortisol test. The same test can be initiated via short range radio directly to the mouthpiece if desired by a web interface connected to a smartphone for signal relay, or it could be initiated by an application on the smartphone or other short-range radio frequency device running Zigbee, Bluetooth, Wi-Fi, or other standard communications protocol. The mouthpiece may have to have an RF transceiver to communicate with for receiving the request to run the test, as well as to send the results of the cortisol test once completed. This mouthpiece could also contain sensors for measuring core body temperature, dehydration, and other indicators that are desirable including the ones rendered by measuring cortisol. All this information could be stored in a central data storage repository in a manner similar to the skull cap and headband designs, and made available via a series of online reports or used to send out text message (SMS), or email alerts when parties need to be notified.

Cortisol sensor implementations utilizing sweat could be placed on other parts of the body for collection of sweat and testing as well. In those scenarios, the test would likely have to have self-contained power from a battery or other source, and would likely have to have short or long-range RF communication capability to know when to perform the test as well as be able to send out the results from such a test. The RF in use could be any “short range” radio including but not limited to Bluetooth, Zigbee, and Wi-Fi. The RF in use could also include GSM, CDMA, TDMA, 3G, 4G, LTE, WiMAX, or any other “long-range” wireless communications protocol in use. Such “long-range” mechanisms would likely have to work and be designed much like a cellular phone is today, and may include an LCD for display, SIM card for network permissions, a power source, antenna, and sensors that can further assist with the mechanism.

Another advancement of the skullcap or headband design would be to include a technique called functional near-Infrared scanning (f-NIR). This technique would involve placing infrared light sources as sensors around the circumference of the skull in a pattern that can allow the light sources to be able to penetrate into all regions of the brain that control cognitive and other functional aspects of the brain associated with movement, memory, coordination, and other cognitive thought processes. If a preferably halo pattern of infrared emitting light sources is implemented around the band portion of the cap or the headband itself, and possibly throughout the upper half of the skullcap, then the light sources could when desired then send light through the brain and have the reflected light waves detected and recorded for purposes of creating an image of the brain activity (blood flow and synaptic impulses). The ideal detector layout would be to have four detectors placed around each infrared light source approximately ½ to 1 inch from the infrared light source itself. To gain maximum benefit from the light source, it is preferably somewhat linear or directional in its light emission beam, but it should disperse enough so as to have reflected light return in a direction that would only be picked up by the intended detectors surrounding that particular sensor once the light is reflected back out from the brain. The pattern for each light source would be to have a single infrared light source closely surrounded by a square pattern of four detectors. This would provide optimal detection of the reflected signals.

Once the reflected light is detected, it can then be mapped into a digital 2D or 3D image of brain activity as desired. This process would be beneficial to the existing skullcap implementation in several ways. One, it would provide another way to look at, understand, and identify a TBI during physical activity or at rest. Two, it would provide a non-radioactive and highly repeatable means by which to monitor patients for certain neurological and cardiological disorders. The same near-Infrared implementation could be made into a shirt or vest that could target monitoring the heart in real-time in the same manner as described for brain activity monitoring. Either implementation could use the accelerometers, heat, and other sensors to use as a trigger and communicate with the other sensors through the control panel on the headgear or through RF. The RF in use could be any “short range” radio including but not limited to Bluetooth, Zigbee, Wi-Fi. The RF in use could also include GSM, CDMA, TDMA, 3G, 4G, LTE, WiMAX, or any other “long-range” wireless communications protocol in use. Such “long-range” mechanisms would likely have to work and be designed much like a cellular phone is today, and may include an LCD for display, SIM card for network permissions, a power source, antenna, and sensors that can further assist with the mechanism. The nearInfrared imaging system could be invoked remotely via an Internet-enabled interface such as a website, hardware or software application, and could be performed as single scan or as multiple scans on a certain time interval.

This would be useful in cases of Epilepsy where the patient is suspected of having a potential seizure due to the accelerometers indicating shaking, and then can be scanned with the near-Infrared to confirm the seizure, or have the imaging mechanism take “snapshots” of the brain function for the next several minutes so the system can record the seizure in real-time as it occurs. This information would be useful to notify others (such as physicians, parents, teachers, coaches, etc.) that a seizure is occurring and they can then take appropriate action to assist the patient. This would also be useful for constant brain activity monitoring as the mechanism is lightweight, inexpensive, and could offer researchers and medical professionals never-before seen recorded views of brain activity across all neurological disorders, which should create a better understanding of such disorders and possibly improve overall quality of care and life for the patient. The cortisol sensors could also be used to initiate or manage when brain scans occur for the person, as certain events such as dehydration, stress, and TBI may warrant performing a brain imaging scan for the person via the headgear during normal activities.

All electronics could be used in various forms of headgear, including helmets, hats, baseball caps, protective head form that includes material designed to absorb shock, ballistics gel, or any other desired material placed on the head. For cheerleading, appearance is considered part of scoring in competition. Therefore, an optimal design would be one that minimizes its appearance for the wearer. Since the control panel is placed at the back base of the skull, it may be hidden under the hair or a pony tail. The sensors then can be run around the sides and top of the hair along wires under or over the hair that can serve to hold the sensors in their appropriate locations and keep the sensor headgear mounted on the head. The sensors around the side of the head can loop over the ears from behind with small wires or see-through material so that they are not visible but can hold the electronics on the head during activities. The headgear can also have a clear plastic or other material that goes around the hairline in the front that may not be visible to others.

In addition to detecting sports injuries and monitoring vital signs during physical activity, the same headgear can be used to monitor for “shaken baby syndrome”. If the skullcap, headband design, etc. is constructed to fit infants or small children, then it can be used for this purpose. The overall headgear logic can use the heat sensor to determine if the headgear has been removed during use to provide tamper detection. The accelerometers can be programmed to detect any shaking of the baby in an undesirable capacity by detecting any G-Force applied to the baby's head beyond a certain threshold. As an example, the accelerometers can be programmed in a manner that may send out alerts or provide an audible alert when the baby's head experiences a G-Force movement of 20 Gs or more as a single event or monitor for repeated events that may indicate abusive or negligent behavior. The alerts can potentially be sent through the wireless transmission network described above to be stored in a centralized data storage facility and then notify any interested parties of the activity. The alerts can also be sent to local electronics receivers (mobile phones, tablets, computers of other types) to act as interfaces to receive the updates. The system can act as a behavior recording device to monitor how caregivers (nurses, babysitters, etc.) are handling the baby or child over an extended timeframe, as well as notify any parties (physicians, hospital administrators, parents, etc.) of the potentially abusive activity the baby or child is being exposed to. The appropriate authority can then take action to correct the situation to safeguard the child.

A flowchart of the transmission of information detected by the present invention can be seen in FIG. 2. The sensor array 20 (not shown) is placed into a skull cap 30 which can be placed, in one embodiment used for a football player, underneath a football helmet 50. Upon impact or forces to the head of the wearer, the sensor array 20 will record the linear and/or rotational forces to the head, and can transit such information by either short or long range mobile transmission 40 to a network computer 60, which is adapted to collect and compare data from a plurality of individuals with sensor arrays to determine and update TBI force thresholds. Such updating can be done in real time. If it is determined that an individual has reached the thresholds for suffering a TBI, an alert can be sent over the internet 70 to either an online TBI care facility database 72, a particular team database 74 that the injured individual works for, or a cell phone or other portable electronic device 76 of a parent or other guardian of the injured individual. Alternatively, the network computer 60 can analyze in real time the frequency of impacts to a given individual, and update the required force thresholds in order for that individual to suffer a TBI based on his history of injury. Further discussion of the connection between the sensory array in the headgear, a network computer, and a central data repository appears in U.S. Non-Provisional application Ser. No. 13/219,605, filed Aug. 27, 2011, which is specifically incorporated by reference herein.

We should also consider various microphone technologies in the design of this audio/vibration detection system. One microphone type that may be considered is the electret condenser microphone, which is the primary type of microphone used in cell phones, PDAs and computers. Electret condenser microphones have historically been considered low-quality and are therefore very inexpensive, but newer models are achieving quality in noise reduction and clarity that rival high-quality microphone types. Piezoelectric microphones are another option to consider. They are considered low quality in the audio world, but they do work well in challenging environments such as under water or high pressure and can pick up vibrations very well, so they may provide good performance for what we are trying to detect. One aspect of piezoelectric microphones is that they rely on mechanical coupling to detect audio signals, which may make them less desirable than other options. Another microphone type that should be considered is fiber-optic. Fiber-optic microphones are very high-quality and should easily detect the audio signatures we are interested in, but are also considered expensive when compared to other microphone technologies. All three microphone technologies discussed should be reviewed and considered for use in this system.

Once an athlete or soldier has a head impact that results in a diagnosed TBI, we may want to further analyze the audio signatures produced at the time of impact. We may want to classify linear impacts that are more consistent with creating the brain bruising, as well as rotational impacts that are more consistent with tissue shearing. Once audio signatures are identified as specific to TBI damage, all information should be reviewed by a medical panel as part of a published medical report to substantiate the findings. Once audio patterns produced by a TBI are known, then we can have microphones manufactured that can only listen on the specific frequency ranges these sounds occur on. In doing so, this can give us the benefit of mechanically removing any other sounds, or audio noise, like the sound of helmets crashing together or other sounds generated around the time of the event. This can dramatically improve the overall performance of the system. Another technique we may want to employ to improve the accuracy of the system is to incorporate noise cancellation to eliminate as much signal interference at the time the impact is recorded to further improve the accuracy of the signal analysis.

The microphone and/or other vibration sensing mechanism for TBI detection can be added to our existing biometric headgear design to enable a comprehensive and possibly Internet-based wearable TBI detection system. Our existing biometric headgear was implemented in two ways for use. One is a skullcap that can fit under helmets for military or sports activities. The other is a headband to use in sports or physical activities that don't utilize protective headgear such as soccer and baseball. Other designs have been described in this and other associated patent disclosures mentioned herein. All may incorporate accelerometers to monitor G-force impact in a manner consistent with the most accurate head impact research design currently available. This research accelerometer design is known as Six Degrees of Freedom (6DOF). Both may also include a heat sensor to monitor for overheating. In the preferred embodiment of the present invention, the biometric headgear implementation uses 6 accelerometers and 1 heat sensor to provide measurements. However, the biometric headgear control panel was designed to support up to 24 sensors in a plug-in and run fashion, so we can easily use the existing design for this new microphone based detection device.

The reason why we prefer to use our existing design is that it already has the wireless technology and control panel finished, and it already has the accelerometer design completed. We also looked at the implementation details of the microphone implementation and realized that we didn't want to send a constant stream of audio and/or vibration recording over wireless networks or have to provide server-side hardware to support such constant streaming of data, both of which would make the service very expensive per person to provide. We instead have decided to utilize the accelerometers to act as a “trigger” to turn on the microphones at say, 30 Gs of shock. We believe that there is a delay of up to milliseconds between when the accelerometers can detect the shock and when the resulting TBI creates any sounds and/or vibrations, which can preferably give us plenty of time to have the accelerometers turn on the microphones and/or other vibration sensors and start recording. Once we start recording, we may only need to record a few seconds of audio signals or other vibrations to capture any sounds and/or vibrations the TBI created. This can give us a low-power solution that may only send small packets of data over wireless networks when impacts of 30 Gs or more occur. Of course, the microphone and/or vibration implementation should be highly accurate if designed properly.

However, the accelerometers can act as an additional filter because only small subsets of audio signals and/or vibrations can be recorded right after a significant impact. This may allow us to better target analysis for correlation with specific audio signatures and further improve the accuracy of TBI detection. As the system is used in the field, the audio and/or vibration recording equipment can work to further identify sounds associated with TBI that may have not been identified in the initial testing and development of the system. As those acoustical and/or vibration patterns are further correlated to actual injury by susceptibility weighted imaging (SWI), MRI, and/or CT scans or further diagnosis by a physician after the injury has occurred, the system can be matured over time to a high degree of accuracy in detecting the injury during physical activity.

The same mechanism of detecting vibration or acoustical signals from the brain as mentioned above can be used in a number of different ways. For instance, all neurological disorders/diseases (Alzheimer's, Parkinsons, Crohn's, Epilepsy, Muscular Sclerosis, etc.) as well as CTE and TBI have a degenerative nature over time. This degenerative nature may cause structural and/or chemical changes in the brain, neural networks, cerebrospinal fluid, grey matter, spinal column, and other neurological tissues and/or surrounding fluids that make up the nervous system. This mechanism could be used to track these degenerative properties by recording vibrations and/or acoustical patterns of the sounds produced in a human subject's head during normal daily activities such as walking or during controlled movements such as swaying the head front-to-back and side-to-side in a reproducible manner. These patterns could be recorded at given intervals during a human subject's life to monitor for the degeneration and/or regeneration of the nervous system. In turn, the same system could also be used to monitor for specific events such as mini-strokes which are events that aren't debilitating in nature, but act as indicators that a major stroke is imminent. In addition, there may be vibration, acoustical signals, or other vital sign changes that occur prior to a seizure, epileptic or otherwise, that may act as an early warning that a seizure is onset or that a seizure is imminent. In Muscular Sclerosis, the onset of an exacerbation as well as monitoring of the condition and recovery from an exacerbation may also be detected by such a mechanism. The degenerative nature of cardiovascular diseases/illnesses could be monitored by the same headgear, similar sensors mounted to the torso or chest area, or any combination of both to detect anomalies and predict or act as early warning mechanisms for heart attacks and other life threatening events as well.

All injuries to a living organism involve tissue being damaged. Tissue tearing and/or breaking, whether involving soft and/or hard tissue, results in the production of vibrations, acoustical or otherwise. These vibrations can be detected by the sensor implementations discussed herein to offer identification of an injury. These vibrations may indicate the extent of an injury as well. Specific vibration patterns may also be detected by sensors to detect certain illnesses. Natural vibration analysis from physiological processes can allow medical personnel to detect and treat a number of illnesses and injuries. Vibration analysis as described herein can also support monitoring of physical and/or mental injury and/or illnesses in any living organism, especially humans. However, the same mechanism can also be used to provide such benefits to livestock, pets, or any animal of interest. This sensor implementation can be used to detect tissue damage and/or illnesses in the human torso, brain, or other parts of the body of interest.

The headgear could incorporate a multi axis accelerometer in combination with a multi axis gyroscope and/or a multi axis motion sensor to achieve specific DOF levels. DOF means degrees of freedom, or number of unique measurements that are used to calculate movement. The most desired DOF for measuring head acceleration during impact is 6DOF. If a motion sensor is used, it can also be used to program in logic that may allow the user to turn the electronics on or off. If the motion sensor is used to turn the electronics on or off, the user would have to move the headgear in a specified manner as to trigger on or off. Such movement patterns could include turning the device over on a table or other flat surface, letting rest for some specific period of time, and then turning the device over again on a table or other flat surface and waiting again for some specified period of time. The controlled pattern can be any number of specific movements and/or time delays that would allow programming logic to know when to turn the device on or off.

The headgear could use direction cosine matrix methods to calculate rotational acceleration from linear acceleration measurements. This could be done in real-time on the device or server side. The headgear could also use Euler angle methods to calculate rotational acceleration from linear acceleration measurements. This could be done in real-time on the device or server side.

The headgear could also use quaternion multiplication methods to calculate rotational acceleration from linear acceleration measurements. This could be done in real-time on the device or server side. Use of quaternions to parameterize rotations leads to numerically well-conditioned systems in the applications under consideration, but incurs an overhead in efficiency and/or code complexity whenever derivatives are used for control or optimization. Especially in light of recent developments, however, they may be the best choice for interpolation of three DOF rotations.

The headgear could be comprised of more than one material and may or may not have holes or channels for airflow. Such headgear may be constructed in a manner where there is preferably a thin outer and inner rigid layer with a less rigid layer in between. The middle layer is preferably able to compress and/or deform as impacts are received to divert force and dampen the actual impact level transferred to the head. This middle layer may have the ability to reshape itself into the original form once the impact force subsides, thus allowing it to return to the original shape or near original shape after impact. This headgear may have the existing electronics located inside to assist in measuring vital signs and identifying TBI or other injuries. The headgear may have a strap that runs under the chin that holds the headgear on. The headgear may also be crafted with a bib in the front much like a baseball cap to block sunlight from the wearer's eyes or shield from rain or other precipitation. Such a headgear design should not have any protruding exterior commonly seen in the back of bicycle helmets. In addition, the headgear would ideally be capable of compressing and/or deforming at low velocity impacts (for instance, under 40 Gs of force applied) as well as demonstrate similar shock absorption properties at higher G force levels (G forces in the 200-300 G range).

Since the measurement sensors may be mounted to a human head with a skullcap, headband, or other material, it is important to understand and factor in any aspects that may impede accurate measurements. The fact that sensors may not stay in contact with the skin throughout an impact may require further consideration and possible error correction. For instance, when the sensors are positioned inside a headband, skullcap, or other cranial attachment via an organic or synthetic material, the material may allow the sensors to move away from the skull during impact. This allowance may dampen or change the true movement of the skull. Since some materials may allow more movement during impact than others, it is important to measure the distance moved during impact as a potential baseline or normalization component and use that distance in calculating any acceleration or rotation measurements the sensors may provide for assessing potential injury or illness. One such adjustment could be to indicate that the material in use is a skullcap made of a specific synthetic or organic material, and based on previous testing, a correction measurement could be used to calculate linear and rotational acceleration based on the elasticity or allowed movement of such material. Each material that is used to hold the sensors against the head or other body part could have a different measured obfuscation or alteration of the true measurements which could be recorded and used for calculating accurate results in various applications.

The sensor device can be programmed to work in a specific manner. This specific manner could be specific to the individual wearing the device, or in a manner consistent with the latest research for detecting injuries or illnesses, or monitoring specific vital sign patterns. This programming of a device could occur as a result of user input from a website or smartphone, etc. interface. The device could also be programmed based on server-side calculations based on defined rules. For instance, if the threshold for TBI is based on frequency of impact at a certain level for monitoring TBI damage occurring sub-concussive impacts, then the server logic could decide that the user needs to have linear or rotational impact thresholds set to a lower level to safeguard an individual player based on their own impact history or overall performance

The tissue damage detection method may also be used to modify performance thresholds for a specific player. For instance, once someone has sustained an injury and the injury is detected by the headgear, the impact readings may be used to reduce the player's threshold of alerts by the system for future participation in sport and/or other physical activity. It is an accepted observation that once a person has a TBI, even after recovery they are more likely to have another TBI. This may indicate that their tolerance under certain levels of force may diminish over their lifetime. If that is the case, then the system can factor that into any thresholds set for a specific individual, and thus notify the individual of a possible TBI at a lower threshold of linear or rotational forces. This should serve to better protect people that may be more susceptible to future TBI or other injuries. The same tissue damage detection method may incorporate pressure, tactile, or other sensors to detect any pressure changes in the skull cavity that are normally associated with epidural, subdural, and/or subarachnoid bleeding, and/or Intraparynchemal Hemorrhaging and/or Tentorial Herniation. Such bleeding could result from a TBI and could build up pressure anywhere inside the skull over time or be apparent shortly after the impact. The detection mechanism may be able to indicate the blood flow created in the skull cavity of some forms of TBI based on hearing or pressure sensing the blood flow as it occurs, or detect any other tissue structural changes associated with such injuries and/or illnesses. The same detection system could also potentially detect damage associated with the spinal column in injuries and illnesses, not simply brain injury or illness, and/or any other neurological injury or illness. Cardiovascular injuries and/or illnesses can also be benefitted by detecting new blood flow and/or pressure changes in blood flow and/or tissue arrangement associated with such injuries and/or illnesses.

Any vital sign or injury/illness monitoring system could have the electronics powered by battery and/or a number of other power sources including but not limited to fuel cell, solar, or other power supply whereby storage of the energy may or may not be used. In addition, one power source could also be incorporating ball bearings or other material that could travel or slide through a chamber on the headgear that could generate mechanical energy, electrical current, or some other form of energy. This energy could be used to directly power the electronics, or used to recharge a battery or other power storage mechanism that could be used to power the electronics. Another power source for the electronics could be body heat or other available power source external to the electronics that could be harnessed to provide power to the electronics.

In addition, the person suspected of injury may also have an additional diagnostic test run that involves cameras placed on the head for analysis. The cameras could be provided as part of a set of goggles or other head mounted apparatus, or as a device that the person places their head into for testing. The cameras can be used to measure pupil dilation, reaction time and ability to certain controlled external stimuli, ability to control motor skills such as blinking, and any other factors that may allow for diagnosis of injury or illness. The camera can take readings and/or record actual video of the person and have that information sent to a physician for review and diagnosis. The physician may be on site or located in a remote location. The communication of data to and from the person may be facilitated by a computer network such as the internet or telco system, and may involve one or more computers on each end or otherwise to view information the cameras are recording. The physician may also interact with the person being evaluated via a telehealth or online care system to facilitate such diagnostics and facilitate providing remote care to the person being examined if it is needed. All information can be recorded as part of the case created in the primary telehealth and online care system. Such information is detailed in U.S. Provisional Application No. 61/719,499 filed Oct. 29, 2012, and is incorporated by reference herein.

The biometric headgear works with a TBI research platform that tracks identification, detection, care/recovery, and reintegration of the patient. The Internet-enabled system tracks data from biometric events and user (physician, patient, parent, coach, and educators) input. The system also stores and provides reports for all the NINDS Common Data Elements for TBI per injury from physicians and athletic trainers during the diagnosis and recovery phase. The system architecture is fully compliant with the latest Oracle/Sun Microsystems JEE Specification and uses the latest and most advanced techniques for web interfacing, data caching, overall system design, etc. The system was designed to be highly scalable and support millions of users if desired. It can also easily accommodate additional data points and workflows as desired for both the biometric research and the telehealth services.

The telemedicine/telehealth industry has traditionally been limited to providing videoconferencing and “store and forward” models for review of medical information between rural clinics and medical experts located some distance from the patient being treated. Primary benefits of this model have been to allow patients to receive treatment from medical experts remotely without the hassle and cost of travel or significant time off from work. This also helps to reduce the number of hospital visits and to lower overall healthcare costs from healthcare providers and insurance companies. However, these systems haven't yet incorporated the added time and cost savings to the patient in providing home care as part of the model, or in providing specific software application services that can dramatically enhance current and future care/recovery models. To date, there has been no telemedicine/telehealth program specifically targeted to provide rehabilitation/reintegration services as part of the overall care model for traumatic brain injuries (TBI). The Archetype TeleHealth 2.0 TBI Platform is designed to address these issues while providing enhancements over historical telehealth initiatives as part of a comprehensive telehealth care model for TBI.

The telehealth care plans used by this system can allow for communications with all appropriate stakeholders in the care and recovery process. The online telehealth services should walk all parties through a step-by-step process to provide care and recovery as well as provide feedback and tracking of a prescribed recovery/reintegration plan once the player is released. This reintegration process is discussed in U.S. Non-Provisional application Ser. No. 13/219,605, filed Aug. 27, 2011, and is specifically incorporated by reference herein. The primary care physician and other medical personnel including neurophysiologists and neuropsychologists, athletic trainers, behavioral workers, social workers, etc. may be able to provide communications with patients, parents or guardians, coaches, educators and teachers, and other parties to maximize care, recovery, and reintegration into normal daily activities whether physical or mental in nature. There is a preferably five-step process for gradual reintegration into physical activity, comprising: light aerobic exercise; moderate exercise; non-contact exercise; resuming practice; and resuming play. These steps are described in further detail in U.S. Provisional Application No. 61/595,195, filed Feb. 6, 2012, and are incorporated by reference herein. The “Return to Play Progression” process is best conducted through a team approach and by a health professional who knows the athlete's physical abilities and endurance level. By gauging the athlete's performance on each individual step, the physician may be able to determine how far the athlete can progress on a given day.

The online care management system provided as part of this system will have a strong focus on managing, and recording “return-to-play” techniques and plans for review and analysis. The primary purpose of the online care management system should be to provide a step-by-step process so physicians, neuropsychologists, and athletic trainers can provide care in an easy to follow online process, while facilitating communications and educational material along the way to promote proper care. Another purpose of such a service is to facilitate TBI and other injury and illness care by supporting proper communications between all stakeholders. Yet another purpose of the system should be to track the measures of care provided for a healthcare professional providing care so that they can verify that adequate and proper guidance was provided as part of the care and recovery/maintenance process. By recording what physicians prescribe and how well the person recovers from the injury or manages the illness, practitioners can be guided toward adoption of best recovery/maintenance strategies for future patients.

It is critical for the physician to guide the patient in their recovery with an active management plan based on their current symptom presentation, even after the patient is released from the physician's care. Careful management can facilitate recovery and prevent further injury. Checklists which can be provided by a physician for monitoring and caring for a patient in recovery can be seen in U.S. Provisional Application No. 61/719,499 filed Oct. 29, 2012, and are incorporated by reference herein. Patients preferably should not return to high risk activities (e.g., sports, physical education (PE), high speed activity (riding a bicycle or carnival rides), if any post concussion symptoms are present or if results from cognitive testing show persistent deficits. When symptoms are no longer reported or experienced, a patient may slowly, gradually, and carefully return to their daily activities (both physical and cognitive). Children and adolescents may need the help of their parents, teachers, coaches, athletic trainers, etc. to monitor and assist with their recovery. Management planning should involve all aspects of the patient's life including home life, school, work, and social or recreational activities.

Increased rest and limited exertion are important to facilitate the patient's recovery. Physicians should be cautious about allowing patients to return to driving, especially if the patient has problems with attention, processing speed, or reaction time. Patients should also be advised to get adequate sleep at night and to take daytime naps or rest breaks when significant fatigue is experienced. Symptoms typically worsen or re-emerge with exertion. Let any return of a patient's symptoms be the guide to the level of exertion or activity that is safe. Patients should limit both physical and cognitive exertion accordingly. Physical activity includes PE, sports practices, weight-training, running, exercising, heavy lifting, etc. Cognitive activity includes heavy concentration or focus, memory, reasoning, reading or writing (e.g., homework, classwork, job-related mental activity). As symptoms decrease, or as cognitive test results show improvement, patients may return to their regular activities gradually. However, the patient's overall status should continue to be monitored closely.

Symptomatic students may require active supports and accommodations in school, which may be gradually decreased as their functioning improves. Inform the student's teacher(s), the school nurse, psychologist/counselor, and administrator of the student's injury, symptoms, and cognitive deficits. Students with temporary yet prolonged symptoms (i.e. longer than several weeks) or permanent disability may benefit from referral for special accommodations and services, such as those provided under a Section 504 Plan, which are implemented when students have a disability (temporary or permanent) that affects their performance in any manner. Services and accommodations for students may include environmental, curriculum, methodology, organizational, behavioral, and presentation strategies. School personnel should be advised to monitor the student for various signs of traumatic brain injury, including increased problems paying attention/concentrating, increased problems remembering/learning new information, longer time required to complete tasks, greater irritability, or less tolerance for stressors. Physicians and school personnel should monitor the student's symptoms with cognitive exertion (mental effort such as concentration, studying) to evaluate the need and length of time supports should be provided.

Guiding the recovery of individuals of any age with mild traumatic brain injury (“MTBI”) who participate in competitive or recreational activities requires careful management to avoid re-injury or prolonged recovery. Athletes engaged in collision sports require special management and evaluation to ensure full recovery prior to their return to play. An individual should not return to competitive sporting or recreational activities while experiencing any lingering or persisting MTBI symptoms. This includes PE class, sports practices and games, and other high-risk/high-exertion activities such as running, bike riding, skateboarding, climbing trees, jumping from heights, playful wrestling, etc. The individual should be completely symptom free at rest and with physical exertion (e.g., sprints, non-contact aerobic activity) and cognitive exertion (e.g., studying, schoolwork) prior to return to sports or recreational activities.

Along with parent and teacher observation for continuing signs or symptoms of concussion, objective data in the form of formal neuropsychological testing may provide valuable information to assist with return to play decisions in younger athletes, as their symptom reporting may be more limited and less reliable. Formal neuropsychological testing of competitive athletes may also help physicians with return to play decisions, as athletes may minimize their symptoms to facilitate return to play. It is important to inform the athlete's coach, PE teacher, and/or athletic trainer that the athlete should preferably not return to play until they are symptom-free and their cognitive function has returned to normal, both at rest and with exertion.

Return to play should occur gradually. Individuals should be monitored for symptoms and cognitive function carefully during each stage of increased exertion. Patients should only progress to the next level of exertion if they are asymptomatic at the current level. Patients with MTBI, particularly during the early post-injury phase, may have difficulties communicating with a physician. Obtaining an accurate report from the patient about the injury and its symptoms with tools such as the ACE is critical to proper management.

In addition to communications problems, it is also important to note that patients may be sensitive to environmental stimuli, such as bright lights, complex visual stimuli such as busy carpet patterns; and/or noise, including from radio or TV. To address this, physicians should consider offering patients access to a quiet, low stimulation waiting area if desired.

There are two types of mental status exams (“MSE”) for detecting neurological injuries—informal and formal. The informal MSE is usually done when clinicians are obtaining historical information from a patient. The formal MSE is performed in a patient suspected of a neurological problem. The patient is commonly asked his/her name, the location, the day, and date. Retentive memory capability and immediate recall can be assessed by determining the number of digits that can be repeated in sequence. Recent memory is typically examined by testing recall potential of a series of objects after defined times, usually within five and 15 minutes. Remote memory can be assessed by asking the patient to review in a coherent and chronological fashion, his or her illness or personal life events that the patient feels comfortable talking about. Patient recall of common historical or current events can be utilized to assess general knowledge. Higher functioning (referring to brain processing capabilities) can be assessed by spontaneous speech, repetition, reading, naming, writing, and comprehension. The patient may be asked to perform further tasks such as identification of fingers, whistling, saluting, brushing teeth motions, combing hair, drawing, and tracing figures. These procedures can assess the intactness of what is called dominant (left-sided brain) functioning or higher cortical function, which refers to the portion of the brain that regulates these activities.

In addition to the care templates above, the physician may want to prescribe specific recovery/reintegration plans as follows. These plans are example templates and the parties involved may want to customize the reintegration strategy based on the individual injury and ability of the person to recover. They may need to last for weeks or months whereby the patient, parent, coach, athletic trainer, teacher/education, etc. may need to follow the plan and report back at specified points to the primary care physician what they are observing, positive and negative, regarding the recovery and reintegration of the patient. All this information should be recorded in a centralized data repository for use in the future by researchers and other governing bodies to develop and mature care/treatment, recovery/reintegration, and overall standardization of care for TBI and other neurological and cardiovascular conditions and injuries.

Another need of the care engagement system is to support an Internet enabled communications forum for anyone dealing with an ongoing health concern such as TBI and other permanent injuries, other neurological disorders, as well as other cardiovascular disorders. Such a forum should support via computer or mobile phone allow users to post questions to other users and physicians for response, as well as provide a means for users to connect and create relationships with people with similar conditions for peer support. These relationships can allow the users of the service to communicate anonymously if desired. The service should be designed in a way as to allow users to link in sensitive electronic medical records to their account, but at the same time shouldn't require users to provide any distinguishing information regarding their identity. In doing so, the service could maintain anonymity even if the site that hosted the service incurs a security breach externally such as “being hacked” or internally by people providing support for the forum.

One such implementation that could support this is to have an application that is designed and developed to run as a Facebook.com plugin, but wouldn't require the personal identification information to become part of the basic requirement of the service. This could mean that each user is simply assigned a serial number to uniquely identify the account with internally when a user sets up a new account. The unique ID could then be used to link login information with an account profile that can link all users and/or physicians and/or support workers (behavioral experts, social workers, etc.) the individual “likes” and “dislikes” for future communications. That same account profile could store or attach to electronic medical records that the owner of the account uploads into the system that provides the details and care history of their condition without providing their name and/or other identifying information. A current Facebook feature that supports this could be the “Document Display” service. Physicians can also provide video to patients for educational purposes as part of the service. Such a forum could be used to support mass communication for a particular type of injury or medical condition in a way never before supported, where users arc free to discuss their injury or illness with others in a truly anonymous capacity without risking divulging their identity to unknown third parties. Likewise, if a participating physician wants to promote a new care plan or provide information to targeted groups participating in the forum, the service should support publishing of information and notices to reach such targeted communities.

There are many benefits and improvements to patient care provided by the TeleHealth 2.0 TBI Platform, including coordinating at-home videoconferencing and Internet enabled services with the primary care physician to significantly reduce hospital and clinical visits; providing education and training to parents, teachers, and coaches to better manage and facilitate care and recovery protocols prior to physician “release;” providing physician-guided reintegration management services to allow all stakeholders to effectively participate in facilitation of the patient back into normal school, sport, and life activities; and incorporating online neurocognitive exams to monitor the patient throughout the recovery and reintegration process.

The telehealth platform is adapted to provide services such as videoconferencing, medical record management, secure messaging, and online appointment scheduling. The TeleHealth 2.0 TBI Platform represents a significant advancement over current telemedicine/telehealth programs in addressing rural care for TBI.

The web enabled vital sign monitoring and injury/illness detection infrastructure may also provide telehealth and online care. Unix is the Operating System used for mission critical networks such as telephone, banking, and military computer networks. The Windows Operating System is the alternative, which can also be used to provide services to users and sensor implementations on the system. The web infrastructure may include a high-performance mapping (web-based GIS with support for street level images, satellite imagery, and even topography) system fully compliant with the Open Geospatial Consortium's (OGC) Web Map Service (WMS) Implementation Specification. This allows the mapping solution to interface with all WMS compliant data sources for mapping. The solution may include overlay support and data for satellite, topography, Nexrad (real time weather) in regions where datasets are available. Additional servers that may be included in the infrastructure are routing (internationalized), messaging (full ESME and UDP capabilities, as well as Orbcomm integration and configuration), database access layer for web based interfaces, reporting (all required reports they need), and monitoring servers to notify support engineers and restart software servers if any hiccup occurs with any of the servers. The engineering support servers may include full recording of system utilization and can even track the number of updates coming into the system and how many users are logged into the system. The TerraTrace platform may record and make available on the Internet full system state and health for support groups to monitor. The system may include a fully integrated billing solution that is fully customizable and supports billing for multiple multi-tiered distribution channels. The entire infrastructure may be a multi-tiered J2EE based infrastructure. The web infrastructure is completely hardware and network agnostic allowing any future advances in hardware technology to be quickly integrated. The infrastructure is currently integrated with telemetry equipment produced by multiple vendors, but we recommend our industry leading solutions (ST, STH, STHO, or STHD).

What is claimed:

1. A system for detecting information related to traumatic brain injury comprising:
a skull cap, adapted to be worn by the individual;
a sensor array coupled to said skull cap comprising:
one or more multi-axial accelerometers, adapted to measure linear, rotational, and/or angular forces;
one or more gyroscopes;
a sensor array computer, coupled to said one or more accelerometers and said one or more gyroscopes, including:
a processor for receiving data, including from said one or more accelerometers and said one or more gyroscopes, pertaining to vital signs of the individual; and
a memory for storing said data;
wherein said sensor array computer is adapted to compare said data to historical data for the individual stored in said sensor array memory to determine if a traumatic brain injury has occurred; and
wherein said historical data includes information relating to the frequency of injuries to the individual's head over a given time period.

2. The system as claimed in claim 1, wherein said sensor array computer is further adapted to update impact thresholds for the individual based on said frequency of injuries.

3. The system as claimed in claim 2, wherein said sensor array computer is adapted to update said impact thresholds in real-time.

4. The system as claimed in claim 2, wherein said sensor array further includes a transceiver adapted to send said updated impact thresholds to a network computer comprising a network computer memory and a network computer processor.

5. The system as claimed in claim 4, wherein said network computer is adapted to compare said impact thresholds with said historical data for the individual to determine if a traumatic brain injury has occurred.

6. The system as claimed in claim 1, wherein said sensor array is fitted inside said skull cap.

7. The system as claimed in claim 4, wherein said transceiver is adapted to operate via one or more of short range wireless transmission and long range wireless transmission.

8. The system as claimed in claim 4, wherein said network computer is a mobile device.

9. The system as claimed in claim 1, further comprising an indication mechanism coupled to said sensor array, adapted to indicate a traumatic brain injury directly to the individual.

10. The system as claimed in claim 1, further comprising one or more cameras in said sensor array, wherein said cameras are adapted to measure a visual factor relating to traumatic brain injury.

11. The system as claimed in claim 10, wherein said visual factor comprises one or more of pupil dilation and reaction time to stimuli.

12. A system for detecting information related to traumatic brain injury comprising:
a skull cap, adapted to be worn by the individual;
a sensor array coupled to said skull cap comprising:
one or more multi-axial accelerometers, adapted to measure linear, rotational, and/or angular forces;
one or more gyroscopes;
a sensor array computer, coupled to said one or more accelerometers and said one or more gyroscopes, including:
a processor for receiving data, including from said one or more accelerometers and said one or more gyroscopes, pertaining to vital signs of the individual; and
a memory for storing said data;
wherein said sensor array computer is adapted to compare said data to historic data for the individual stored in said sensor array memory to determine if a traumatic brain injury has occurred; and
wherein said headgear comprises a plurality of layers, said plurality of layers comprising an outer rigid layer, an inner rigid layer, and at least one softer middle layer in-between said outer layer and said inner layer.

13. The system as claimed in claim 12, wherein said at least one middle layer is adapted to compress upon impact to the head of the individual and to expand to its original shape after said impact.

14. The system as claimed in claim 12, wherein said sensor array is fitted inside said skull cap.

15. The system as claimed in claim 12, further comprising an indication mechanism coupled to said sensor array, adapted to indicate a traumatic brain injury directly to the individual.

16. A system for detecting information related to traumatic brain injury comprising:
a skull cap, adapted to be worn by the individual;
a sensor array coupled to said skull cap comprising:
one or more multi-axial accelerometers, adapted to measure linear, rotational, and/or angular forces;
one or more gyroscopes;
a sensor array computer, coupled to said one or more accelerometers and said one or more gyroscopes, including:
a processor for receiving data, including from said one or more accelerometers and said one or more gyroscopes, pertaining to vital signs of the individual; and
a memory for storing said data;
wherein said sensor array computer is adapted to compare said data to historic data for the individual stored in said sensor array memory to determine if a traumatic brain injury has occurred; and
wherein said sensor array computer is adapted to calculate acceleration from said linear forces.

17. The system as claimed in claim 16 wherein said acceleration is calculated using quaternion multiplication.

18. The system as claimed in claim 16, wherein said sensor array computer is adapted to calculate said acceleration in real-time.

19. The system as claimed in claim 16, wherein said sensor array is fitted inside said skull cap.

20. The system as claimed in claim 16, further comprising an indication mechanism coupled to said sensor array, adapted to indicate a traumatic brain injury directly to the individual.
